(19) United States
(12) Patent Application Publication
(10) Pub. No.: US 2014/0161748 A1
(43) Pub. Date: Jun. 12, 2014

(54) METHODS FOR TREATING SUN-EXPOSED SKIN

(71) Applicants: Xin-Min Li, Edmonton (CA); Jiming Kong, Winnipeg (CA); Handi Zhang, Winnipeg (CA); Kelly Hartle, Winnipeg (CA)

(72) Inventors: Xin-Min Li, Edmonton (CA); Jiming Kong, Winnipeg (CA); Handi Zhang, Winnipeg (CA); Kelly Hartle, Winnipeg (CA)

(73) Assignee: UNIVERSITY OF MANITOBA, Winnipeg, MB (CA)

(21) Appl. No.: 14/116,450

(22) PCT Filed: May 10, 2012
(86) PCT No.: PCT/IB2012/052341

§ 371 Date: Feb. 24, 2014

Related U.S. Application Data
(60) Provisional application No. 61/485,908, filed on May 13, 2011.

(51) Int. Cl. A61K 31/554; A61Q 17/04; A61Q 19/08; A61K 8/49

(52) U.S. Cl. CPC A61K 31/554; A61K 8/49; A61Q 17/04; A61Q 19/08
USPC 424/59; 514/211.13

(57) ABSTRACT

The present invention includes methods for treating sun-exposed skin, such as treating photoaging and treating skin cancer. Also included are methods for altering a cell's response to ultraviolet radiation. The methods include administration of quetiapine or an analog thereof or a pharmaceutically acceptable salt, solvate, or prodrug thereof.

### CROSS-REFERENCE TO RELATED APPLICATIONS

This application claims the benefit of U.S. Provisional Application Ser. No. 61/485,908, filed May 13, 2011, which is incorporated by reference herein.

### BACKGROUND

Topical application of antioxidants is a promising treatment to prevent damage caused by excess reactive oxygen species (ROS). There are many products with antioxidant properties under investigation including vitamin C, vitamin E, beta carotene, caffeic acid, isoflavones, flavenoids, tea polyphenols, selenium and zinc. These antioxidants often have the undesirable characteristics of instability and cosmetically unpleasing coloration, which make their use difficult.

The current solutions used to prevent or to repair skin damaged by UV radiation include use of sunscreen, protective clothing engineered to filter out UVA and UVB, topical retinoids which are derivatives of vitamin A and have anti-aging properties, injection of Botulinum toxin and soft tissue fillers, and laser procedures. However, each of these solutions have drawbacks. Sunscreens require constant reapplication, not all are cosmetically acceptable which discourages use, and not all protect against both UVA and UVB. Protective clothing can be expensive and may be undesirable to a subject. Use of topical retinoids may result in retinoid dermatitis in a subject, and may not be tolerated well by all subjects. The use of Botulinum toxin, soft tissue fillers, and laser procedures is expensive and also potentially risky. Research into how natural aging and sun exposure influence our skin is a huge area of interest and has advanced considerably in the past two decades. The desire to maintain a youthful look as we age has led an increasing interest in creams and procedures that can improve the appearance of skin. Development of affordable products that can safely and effectively prevent UV damage or that can repair damaged skin would be a major contribution.

### SUMMARY OF THE INVENTION

The present invention represents an advance in the prevention of skin damage from ultraviolet (UV) radiation. Quetiapine is an atypical antipsychotic compound used for the treatment of schizophrenia, acute episodes associated with bipolar disorder, and maintenance treatment of depression and bipolar disorder. The inventors have made the observation that Quetiapine will prevent some of the effects UV radiation has on skin. The observation that Quetiapine possess this activity is surprising and unexpected.

Accordingly, provided herein is are methods for using an active quetiapine compound selected from quetiapine, an analog of quetiapine, and a pharmaceutically acceptable salt, solvate, and prodrug thereof. In one embodiment, the method is for treating photoaging. The method includes administering to a subject, such as a human, in need thereof an effective amount of a composition that includes an active quetiapine compound selected from quetiapine, an analog of quetiapine, and a pharmaceutically acceptable salt, solvate, and prodrug thereof, wherein the active quetiapine compound is effective in treating photoaging. The administering may occur before or during extended exposure of the subject to a source of ultraviolet radiation. In one embodiment, the treating may include reducing redness and/or irritation of the subject's skin after exposure of the subject to a source of UV radiation. In one embodiment, the treating may include minimizing an increase in epidermis of the subject's skin, maintaining uniform and intact collagen bands in the subject's skin, preserving elastic fiber number and structure in the subject's skin, or a combination thereof, after exposure of the subject to a source of UV radiation. The source of UV radiation may be natural sunlight and/or artificial. In one embodiment, the administering may include use of a topical preparation, and may be to a location chosen from the face, legs, arms, and hands. In one embodiment, the administering may be oral. The subject may or may not display a sign of photoaging prior to the administering. In one embodiment, the skin cells of the subject may have greater levels of proCOL1A1 after exposure to a source of ultraviolet radiation than skin cells of the subject not administered the composition and exposed to the source of ultraviolet radiation. In one embodiment, the skin cells of the subject may have reduced levels of MMP1 after exposure to a source of ultraviolet radiation than skin cells of the subject not administered the composition and exposed to the source of ultraviolet radiation.

Also provided herein is a method for treating skin cancer. The method includes administering to a subject, such as a human, in need thereof an effective amount of a composition that includes an active quetiapine compound selected from quetiapine, an analog of quetiapine, and a pharmaceutically acceptable salt, solvate, and prodrug thereof, wherein the active quetiapine compound is effective in treating skin cancer. The administering may occur before or during extended exposure of the subject to a source of ultraviolet radiation. In one embodiment, the treating may include decreasing the severity of one or more signs of skin cancer. In one embodiment, the treating may include preventing one or more signs of skin cancer. The source of UV radiation may be natural sunlight and/or artificial. In one embodiment, the administering may include use of a topical preparation, and may be to a location chosen from the face, legs, arms, and hands. In one embodiment, the administering may be oral. The subject may or may not display a sign of skin cancer prior to the administering. The skin cancer may be a basal cell cancer, a squamous cell cancer, or a melanoma. In one embodiment, the skin cells of the subject may have greater levels of proCOL1A1 after exposure to a source of ultraviolet radiation than skin cells of the subject not administered the composition and exposed to the source of ultraviolet radiation. In one embodiment, the skin cells of the subject may have reduced levels of MMP1 after exposure to a source of ultraviolet radiation than skin cells of the subject not administered the composition and exposed to the source of ultraviolet radiation.

In one embodiment, the active quetiapine compound used in a method described herein is selected from a compound of Formula I, and a pharmaceutically acceptable salt and solvate thereof:

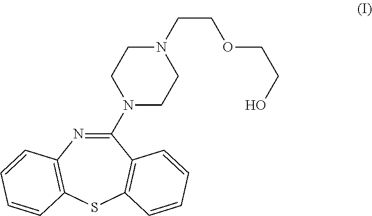 [SMILES: OCCOCCN1CCN(C2=Nc3ccccc3Sc3ccccc32)CC1]

wherein one or more hydrogen-bearing carbon atoms in the quetiapine is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

In one embodiment, the analog of quetiapine used in a method described herein is selected from a compound of Formula II, and a pharmaceutically acceptable salt, solvate, and prodrug thereof:

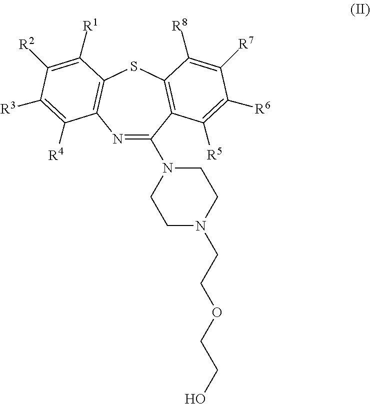

wherein any one or more of R₁, R₂, R₃, R₄, R₅, R₆, R₇, R₈ (e.g., one, two, three, four, five, six, seven, or eight) are independently selected from halogen (e.g., F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), CF₃, OCF₃CH₃, or a C1-C10 organic group (e.g., in some embodiments a C1-C4 organic group or moiety), with each R independently being hydrogen or an organic group.

Also provided herein are uses of an active quetiapine compound selected from quetiapine, an analog of quetiapine, and a pharmaceutically acceptable salt, solvate, and prodrug thereof. The uses may include a use in the preparation of a medicament for skin cancer, or a use for treating skin cancer, wherein the skin cancer includes a basal cell cancer, a squamous cell cancer, or a melanoma. The uses may include a use in the preparation of a medicament for photoaging or a use for treating photoaging. In one embodiment, the medicament may be an oral medicament. In one embodiment, the medicament may be a topical medicament.

In one embodiment, the active quetiapine compound present in a use described herein is selected from a compound of Formula I, and a pharmaceutically acceptable salt and solvate thereof:

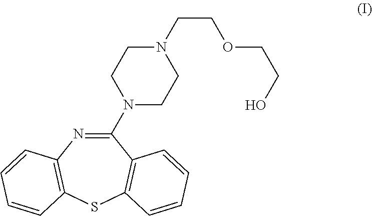 [SMILES: OCCOCCN1CCN(C2=Nc3ccccc3Sc3ccccc32)CC1]

wherein one or more hydrogen-bearing carbon atoms in the quetiapine is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

In one embodiment, the analog of quetiapine present in a use described herein is selected from a compound of Formula II, and a pharmaceutically acceptable salt, solvate, and prodrug thereof:

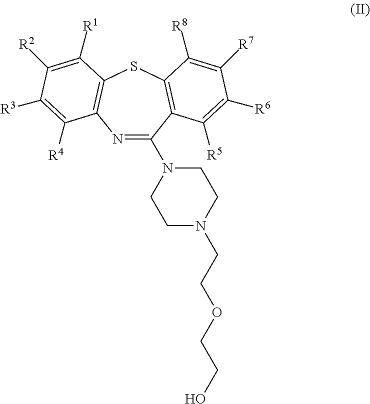

wherein any one or more of R₁, R₂, R₃, R₄, R₅, R₆, R₇, R₈ (e.g., one, two, three, four, five, six, seven, or eight) are independently selected from halogen (e.g., F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), CF₃, OCF₃CH₃, or a C1-C10 organic group (e.g., in some embodiments a C1-C4 organic group or moiety), with each R independently being hydrogen or an organic group.

Provided herein is a composition that includes an active quetiapine compound selected from quetiapine, an analog of quetiapine, and a pharmaceutically acceptable salt, solvate, and prodrug thereof, and a vehicle suitable for use by epicutaneous administration. In one embodiment, the active quetiapine compound present in a composition is selected from a compound of Formula I, and a pharmaceutically acceptable salt and solvate thereof:

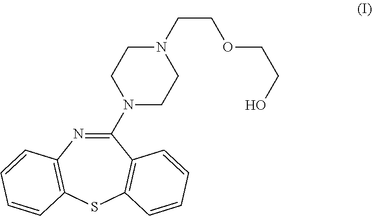 [SMILES: OCCOCCN1CCN(C2=Nc3ccccc3Sc3ccccc32)CC1]

wherein one or more hydrogen-bearing carbon atoms in the quetiapine is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

In one embodiment, the analog of quetiapine present a composition is selected from a compound of Formula II, and a pharmaceutically acceptable salt, solvate, and prodrug thereof:

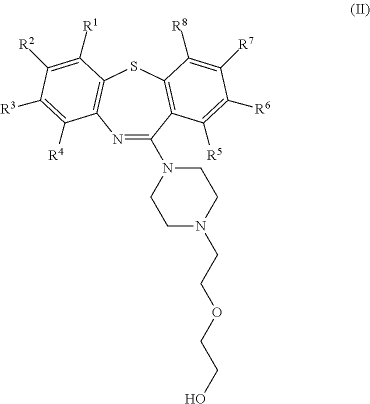

wherein any one or more of R₁, R₂, R₃, R₄, R₅, R₆, R₇, R₈ (e.g., one, two, three, four, five, six, seven, or eight) are independently selected from halogen (e.g., F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), CF₃, OCF₃CH₃, or a C1-C10 organic group (e.g., in some embodiments a C1-C4 organic group or moiety), with each R independently being hydrogen or an organic group.

Also provided herein are methods for altering a cell's response to ultraviolet radiation. In one embodiment, the method includes contacting an ex vivo cell with an effective amount of an active quetiapine compound selected from quetiapine, an analog of quetiapine, and a pharmaceutically acceptable salt, solvate, and prodrug thereof, and exposing the cell to a source of ultraviolet radiation, wherein the amount of proCOL1A1 in the cell after exposure of the cell to ultraviolet radiation is greater than the amount of proCOL1A1 content in a control cell not contacted with the active quetiapine compound and exposed to the ultraviolet radiation. In one embodiment, the method includes contacting an ex vivo cell with an effective amount of an active quetiapine compound selected from quetiapine, an analog of quetiapine, and a pharmaceutically acceptable salt, solvate, and prodrug thereof, and exposing the cell to a source of ultraviolet radiation, wherein the amount of MMP1 in the cell after exposure of the cell to ultraviolet radiation is lower than the amount of MMP1 in a control cell not contacted with the active quetiapine compound and exposed to the ultraviolet radiation. In one embodiment, the cell may be a keratinocyte, a melanocyte, a Langerhans cell, or a fibroblast.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Hematoxylin and Eosin staining of skin samples from mice. The very top fibrous looking layer is the stratum corneum, which includes dead cells. The layer beneath the stratum corneum, appearing the darkest, is the epidermis and below that, is the dermis. A, image of stained normal skin. B, image of stained untreated UV exposed skin. C, image of stained Quetiapine treated UV exposed skin. Magnification is 10×.

FIG. 2. Van Gieson staining of skin samples from mice. Collagen is located immediately underneath the epidermis. Fibres run horizontally through the tissue. A, image of stained untreated UV exposed skin. B, image of stained Quetiapine UV exposed skin. Magnification is 20×.

FIG. 3. Verhoeff-Van Gieson staining of skin samples from mice. Collagen appears as bands running horizontally directly beneath the epidermis. Elastic fibres appear as smaller, darker, bands sprinkled throughout the collagen bands. A, image of stained normal skin. B, image of stained untreated UV exposed skin. C, image of stained treated UV exposed skin. Magnification is 20×.

### DETAILED DESCRIPTION OF ILLUSTRATIVE EMBODIMENTS

Provided herein are compositions that include a compound useful in the methods described herein. In one embodiment such a compound is quentiapine, or a pharmaceutically acceptable salt, solvate, or prodrug thereof. Quetiapine (IUPAC name: 2-(2-(4-dibenzo[b,f][1,4]thiazepine-11-yl-1-piperazinyl)ethoxy)ethanol, available under the trade name Seroquel) has the following structure (Formula I):

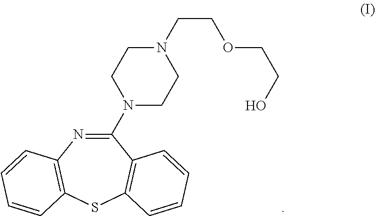 [SMILES: OCCOCCN1CCN(C2=Nc3ccccc3Sc3ccccc32)CC1]

In one embodiment, a composition useful in the methods described herein is an analog of quetiapine, or a pharmaceutically acceptable salt, solvate, or prodrug thereof. In one embodiment, an analog of quetiapine is a compound disclosed by Edgar et al. (U.S. Pat. No. 7,563,785). Analogs include metabolites of quetiapine. A metabolite of quetiapine includes compounds resulting from the metabolism of quetiapine in the body. At least twenty metabolites of quetiapine have been identified (Goren & Levin, 1998, Pharmacotherapy, 18:1183-1194). Examples of metabolites include the 7-hydroxylated metabolite and the N-dealkylated metabolite (Goren & Levin, 1998, Pharmacotherapy, 18:1183-1194), N-desalkylQuetiapine, 7-hydroxyQuetiapine, O-desalkylQuetiapine, and the corresponding sulfoxinde and sulfone analogs and corresponding phenolated analogs (Bakken et al., 2009, Drug Metabol. Dispos., 37:254-258; see also Mickle et al., US Patent Application 20110183963, and Mickle et al., US Patent Application 20110223207).

In one embodiment, an analog of quetiapine is selected from a compound that is the result of one or more substitutions of Formula I, and pharmaceutically acceptable salts, solvates, and prodrugs thereof. The skilled person will recognize that the hydrogen atom on one or more of the hydrogen-bearing carbon atoms in the quetiapine structure can be substituted with a substituent including, but not limited to, halogen (e.g. F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), CF₃, OCF₃CH₃, or a C1-C10 organic group (e.g., in some embodiments a C1-C4 organic group or moiety), with each R independently being hydrogen or an organic group.

In one embodiment, an analog of quetiapine is selected from a compound of Formula II, and pharmaceutically acceptable salts, solvates, and prodrugs thereof:

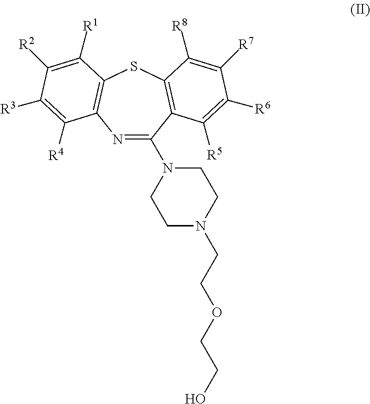

wherein any one or more of R₁, R₂, R₃, R₄, R₅, R₆, R₇, R₈ (e.g., one, two, three, four, five, six, seven, or eight) are independently selected from halogen (e.g., F, Cl, Br, I), nitrile (CN), hydroxy (Oil), alkoxy (OR), nitrate (O—NO₂), nitrite (O—N═O), sulfate (O—SO₃R), amino (NR₂), nitro (NO₂), sulfonate (SO₂OR), CF₃, OCF₃CH₃, or a C1-C10 organic group (e.g., in some embodiments a C1-C4 organic group or moiety), with each R independently being hydrogen or an organic group.

As used herein, the term “organic group” is used for the purpose of this invention to mean a hydrocarbon group that is classified as an aliphatic group, cyclic group, or combination of aliphatic and cyclic groups (e.g., alkaryl and aralkyl groups). In the context of the present invention, suitable organic groups for compounds of this invention are those that do not interfere with the ability of a compound to inhibit an effect of UV radiation on skin. In the context of the present invention, the term “aliphatic group” means a saturated or unsaturated linear or branched hydrocarbon group. This term is used to encompass alkyl, alkenyl, and alkynyl groups, for example. The term “alkyl group” means a saturated linear or branched monovalent hydrocarbon group including, for example, methyl, ethyl, n-propyl, isopropyl, tert-butyl, amyl, heptyl, and the like. The term “alkenyl group” means an unsaturated, linear or branched monovalent hydrocarbon group with one or more olefinically unsaturated groups (i.e., carbon-carbon double bonds), such as a vinyl group. The term “alkynyl group” means an unsaturated, linear or branched monovalent hydrocarbon group with one or more carbon-carbon triple bonds. The term “cyclic group” means a closed ring hydrocarbon group that is classified as an alicyclic group, aromatic group, or heterocyclic group. The term “alicyclic group” means a cyclic hydrocarbon group having properties resembling those of aliphatic groups. The term “aromatic group” or “aryl group” means a mono- or polynuclear aromatic hydrocarbon group. The term “heterocyclic group” means a closed ring hydrocarbon in which one or more of the atoms in the ring is an element other than carbon (e.g., nitrogen, oxygen, sulfur, etc.).

As a means of simplifying the discussion and the recitation of certain terminology used herein, the terms “group” and “moiety” are used to differentiate between chemical species that allow for substitution or that may be substituted and those that do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

As used herein, “solvate” means a compound wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate.” The formation of solvates of the compounds described herein will vary depending on the identity of the compound and the solvate. In general, solvates are formed by dissolving the compound in the appropriate solvent and isolating the solvate by cooling or using an antisolvent. The solvate is typically dried or azeotroped under ambient conditions.

As used herein, “prodrug” means a derivative of quetiapine or a quetiapine analog, designed to undergo either a chemical or biochemical transformation in the subject to release the active compound. Prodrugs of quetiapine or quetiapine analogs may be, for example, conventional esters formed with available hydroxy groups. For example, an available hydroxy group may be acylated using an activated acid in the presence of a base, and optionally, in inert solvent (e.g. an acid chloride in pyridine). Some common esters which have been utilized as prodrugs are phenyl esters, aliphatic esters, acyloxymethyl esters, carbamates and amino acid esters.

Whether quetiapine, an analog of quetiapine, or a pharmaceutically acceptable salt, solvate, or prodrug thereof is useful in the methods described herein can be determined by a suitable in vitro assay (see Example 1) or a suitable in vivo assay (see Example 2). As used herein, the term “active quetiapine compound” refers to quetiapine, an analog of quetiapine, or a pharmaceutically acceptable salt, solvate, or prodrug thereof that is able to (i) prevent skin redness and irritation in 9 week old mice after exposure to OV radiation as described in Example 2, (ii) minimize an increase in epidermis that results after exposure to UV radiation as described in Example 2, (iii) maintain uniform and intact collagen bands in skin after exposure to UV radiation as described in Example 2, and/or (iv) preserve the elastic fiber number and structure in skin after exposure to UV radiation as described in Example 2.

Methods of preparing quetiapine and analogs thereof are reported by Warawa et al., (U.S. Pat. No. 4,879,288), Diller et al. (US Patent Application 20040220400), Etlin et al. (US Patent Application 20060063927), Puig et al. (US Patent Application 20060189594), Edgar et al. (U.S. Pat. No. 7,563,785), and Hradil et al. (U.S. Pat. No. 8,034,805).

A composition including an active quetiapine compound may include a pharmaceutically acceptable carrier. “Pharmaceutically acceptable” refers to a pharmacologically inactive substance that is compatible with the other ingredients of the composition, and not deleterious to the recipient thereof. Typically, the composition includes a pharmaceutically acceptable carrier when the composition is used as described herein.

A composition may be prepared by methods well known in the art of pharmacy. The compositions disclosed herein may be formulated in pharmaceutical preparations in a variety of forms adapted to the chosen route of administration. A formulation may be solid or liquid. Administration may be systemic or local. In some aspects local administration may have advantages for site-specific, targeted management of a condition described herein. Local therapies may provide high, clinically effective concentrations directly to the treatment site, with less likelihood of causing systemic side effects.

Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. A composition for use in topical administration may be formulated into many types of vehicles. Non-limiting examples of suitable vehicles include emulsions (e.g., oil-in-water, water-in-oil, silicone-in-water, water-in-silicone, water-in-oil-in-water, oil-in-water, oil-in-water-in-oil, oil-in-water-in-silicone, etc.), creams, lotions, solutions (both aqueous and hydro-alcoholic), anhydrous bases (such as lipsticks and powders), gels, ointments, pastes, or eye jellies. Variations and other vehicles will be apparent to the skilled artisan and are appropriate for use in the methods described herein.

It is also contemplated that an active quetiapine compound may be encapsulated for delivery to a target area such as skin. Non-limiting examples of encapsulation techniques include the use of liposomes, vesicles, and/or nanoparticles (e.g., biodegradable and non-biodegradable colloidal particles comprising polymeric materials in which the ingredient is trapped, encapsulated, and/or absorbed—examples include nanospheres and nanocapsules) that can be used as delivery vehicles to deliver such ingredients to skin.

A composition intended for oral delivery may include an inert diluent or an edible carrier. For the purpose of oral therapeutic administration, the active quetiapine compound can be incorporated with excipients and used in the form of tablets, troches, or capsules. Oral compositions can also be prepared using a fluid carrier. Pharmaceutically compatible binding agents can be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Compositions described herein may be incorporated into products. Non-limiting examples of products include cosmetic products, pharmaceutical products, etc. Examples of pharmaceutical products include, but are not limited to, tablets. Examples of cosmetic products include, but are not limited to, sunscreen products, sunless skin tanning products, moisturizing creams, skin creams and lotions, softeners, day lotions, gels, ointments, foundations, night creams, lipsticks and lip balms, cleansers, toners, masks, exfoliating compositions, shaving-related products (e.g., creams, “bracers” and aftershaves), pre-moistened wipes and washcloths, tanning lotions, bath products such as oils, skin colorant and make-up products such as foundations, blushes, rouges eye shadows and lines, lip colors and mascaras, and skin or facial peel products. Compositions for use in administration may include the active quetiapine compound at a concentration of at least 0.00001%, at least 0.0001%, at least 0.001%, at least 0.01%, at least 0.1%, or at least 1% by weight of the total composition. If desired, higher concentrations may be used.

Compositions described herein may include additional ingredients. Non-limiting examples of additional ingredients include cosmetic ingredients. Examples of cosmetic ingredients that can be used in the context of the methods described herein include: fragrances, dyes and color ingredients, emulsifiers, stabilizers, lubricants, solvents, moisturizers, water-repellants, UV absorbers, essential oils, vitamins, anti-irritants, botanical extracts, anti-microbial agents, antioxidants, chelating agents, preservatives, and skin conditioning agents.

Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred.

Provided herein are methods for using an active quetiapine compound. In one embodiment, the active quetiapine compound is present in a composition. In some embodiments, a method for using a composition described herein includes treating certain conditions in a subject in need of treatment. In one embodiment, a method includes administering to a subject in need thereof an effective amount of an active compound described herein. The term “effective amount” refers to an amount of an active compound which is capable of achieving a desired effect. The subject may be a mammal, including a member of the family Muridae (a murine animal such as rat or mouse), a primate, (e.g., monkey or human), a rabbit, or a dog. In one embodiment, the subject is a human. As used herein, the term “condition” refers to any deviation from or interruption of the normal structure or function of a part, organ, or system, or combination thereof, of a subject that is manifested by a characteristic symptom or clinical sign. Conditions include damage to skin due to ultraviolet exposure.

As used herein, the term “symptom” refers to subjective evidence of a condition experienced by a subject. As used herein, the term “clinical sign,” or simply “sign,” refers to objective evidence of a condition present in a subject. Symptoms and/or signs associated with conditions referred to herein and the evaluation of such signs are routine and known in the art. Examples of signs of conditions vary depending upon the condition. Signs of skin damage due to ultraviolet exposure include photoaging and skin cancer. Signs of photoaging include, but are not limited to, skin that is wrinkled, lax, and/or coarse; increased epidermal thickness, atrophy of extracellular matrix, impaired organization of connective tissue (e.g. disorganized and/or fragmented appearance of collagen, elastic fibers that are decreased in number and abnormally long and thin), increased redness, increased irritation, and/or premature aging. Examples of skin cancer include, but are not limited to, basal cell carcinoma, squamous cell carcinoma, and melanoma. Signs of basal cell carcinoma include, but are not limited to, a raised, smooth, pearly bump on the sun-exposed skin. Signs of squamous cell carcinoma include, but are not limited to, a red, scaling, thickened patch on sun-exposed skin. Signs of melanoma include, but are not limited to, a brown to black lesion optionally with change in size, shape, color, and/or elevation. Whether a subject has a condition, and whether a subject is responding to treatment, may be determined by evaluation of signs associated with the condition.

Treatment of a condition can be prophylactic or, alternatively, can be initiated after the development of a condition. Treatment that is prophylactic, for instance, initiated before a subject manifests signs of a condition, is referred to herein as treatment of a subject that is “at risk” of developing a condition. An example of a subject that is at risk of developing a condition is a person likely to have extended exposure to a source of UV radiation, such as the sun or an artificial source. Extended exposure to a UV source may be at least 10 minutes, at least 1 hour, at least 5 hours, or at least 10 hours. The administration can be before, during, or after exposure to a source of UV radiation. Treatment can be performed before, during, or after the occurrence of the conditions described herein. Another example of a subject that is at risk of developing a condition is a person having fair skin. Treatment initiated after the development of a condition may result in decreasing the severity of the signs of the condition, or completely removing the signs.

In one embodiment, the method is directed to treating skin damage, such as photoaging, in a subject. In one embodiment, the method is directed to treating skin damage, such as skin cancer, in a subject. In one embodiment, the composition may be administered to the skin of the body, such as the face, legs, arms, hands, or any other skin of the body. In one embodiment, the composition may be administered orally to the subject. In one embodiment, the method may result in reduced redness and/or irritation of the subject's skin after exposure of the subject to a source of UV radiation when compared to skin of the subject not administered the composition but exposed to the source of ultraviolet radiation. In one embodiment, the method may result in minimizing an increase in epidermis of the subject's skin, maintaining uniform and intact collagen bands in the subject's skin, and/or preserving elastic fiber number and structure in the subject's skin, after exposure of the subject to a source of UV radiation when compared to skin of the subject not administered the composition but exposed to the source of ultraviolet radiation. In one embodiment, the method may result in skin cells of the subject having greater levels of collagen and/or proCol1A1 than cells of the subject that were exposed to the UV source but that did not receive the composition. In one embodiment, the method may result in skin cells of the subject having decreased levels of MMP1 compared to cells of the subject that were exposed to the UV source but that did not receive the composition. The cells of a subject demonstrating a change in collagen, proCol1A and/or MMP1 may be present in the epidermis, dermis, and/or subcutaneous tissue of the subject.

The ultraviolet radiation to which a subject is exposed may be UVA (between 320 nanometers (nm) and 400 nm), UVB (between 391 nm and 280 nm), UVC (between 279 nm and 100 nm), or a combination thereof. The source may be natural sunlight, or an artificial source, such as a sun lamp.

In one embodiment the method includes altering a cell's response to ultraviolet radiation. In one embodiment, the method includes contacting a cell with an effective amount of an active quetiapine compound. The cell may be ex vivo or in vivo. As used herein, an “ex vivo” cell refers to a cell that has been removed, for instance, isolated, from the body of a subject. Ex vivo cells include, for instance, primary cells (e.g., cells that have recently been removed from a subject and are capable of limited growth or maintenance in tissue culture medium), and cultured cells (e.g., cells that are capable of extended growth or maintenance in tissue culture medium). Examples of suitable cells include, but are not limited to, a keratinocyte, a melanocyte, a Langerhans cell, or a fibroblast. Other examples of suitable cells include tumor cells, tumor cell lines, and cell lines susceptible to tumor formation. As used herein, an “in vivo” cell refers to a cell that is within the body of a subject. An in vivo cell may be present in the epidermis, dermis, and/or subcutaneous tissue of the animal. An in vivo cell may be a cell present in an organ or a tumor. The cell is preferably a mammalian cell, such as, for instance, mouse, rat, rabbit, dog, or primate (e.g., monkey or human). In one embodiment, the cell is a human cell.

The term “effective amount” refers to an amount of an active quetiapine compound which is capable of achieving the desired effect. In one embodiment, when a cell is exposed to ultraviolet radiation it may respond by decreasing the amount of proCOL1A1 and/or increasing the amount of matrix metalloproteinase 1 (MMP1). In one embodiment, contacting a cell with an effective amount of an active quetiapine compound results in minimizing the decrease in the amount of proCOL1A1. Thus, a cell contacted with an active compound and exposed to a UV source will have higher levels of proCOL1A1 than a control cell exposed to the UV source but not contacted with the active quetiapine compound. In one embodiment, contacting a cell with an effective amount of an active compound results in minimizing the increase in the amount of MMP1. Thus, a cell contacted with an active quetiapine compound and exposed to a UV source will have lower levels of MMP1 than a control cell exposed to the UV source but not contacted with the active quetiapine compound. Methods for measuring proCOL1A1 and MMP1 are known in the art and are routine. Examples include the use of specific antibody and/or nucleic acid-based assays to measure specific mRNA. A change in the degree of reduction of proCOL1A1 content in a cell compared to a control cell, and/or a change in the degree of increasing MMP1 in a cell compared to a control cell indicates the cell's response to UV radiation has been altered. In one embodiment, a cell's response to UV radiation is considered to have been altered if there is a statistically significant change in proCOL1A1 or MMP1 levels compared to a control cell not exposed to the compound. In one embodiment, a cell's response to UV radiation is considered to have been altered if there is a change in proCOL1A1 or MMP1 levels of at least 0.01%, at least 0.1%, at least 1%, at least 2.5%, at least 5%, at least 10%, at least 15%, at least 20%, at least 25%, at least 30%, at least 35%, at least 40%, at least 45%, or at least 50% compared to a control cell not exposed to the compound.

The ultraviolet radiation to which the cell is exposed may be UVA (between 320 nanometers (nm) and 400 nm), UVB (between 391 nm and 280 nm), UVC (between 279 nm and 100 nm), or a combination thereof. The source may be natural sunlight, or an artificial source, such as a sun lamp.

The present invention is illustrated by the following examples. It is to be understood that the particular examples, materials, amounts, and procedures are to be interpreted broadly in accordance with the scope and spirit of the invention as set forth herein.

### Example 1

### Anti-Aging Effect of Quetiapine on Skin

Ultraviolet (UV) radiation causes many acute and chronic detrimental cutaneous effects. Two of the most important consequences are skin cancer and photoaging. Photoaging, caused by UV radiation, essentially speeds up the natural aging process by damaging collagen, the major component of connective tissue in skin. Oxidative stress has a central important role in these processes. Antioxidants have become an important complementary method for traditional sunscreens to protect skin from UV induced damage. The purpose was to examine whether the anti-psychotic drug Quetiapine may protect skin fibroblasts from injury induced by UV radiation via reducing oxidative stress.

Quetiapine's mechanisms of action that make it an effective treatment are fairly well known. The potential for Quetiapine to be used for its anti-oxidant properties is less well known. Part of the drug's effectiveness maybe its ability to inhibit certain pathways involved in cell death and the release of reactive oxygen species and to promote the expression of genes that protect against generators of reactive oxygen species, increase antioxidant defenses, and decrease levels of oxidative damage. It is possible that these properties of Quetiapine may also function to decrease the oxidative damage done to cells after exposure to UV radiation.

### Dose-Cell Viability Effect and Time-Cell Viability Effect of UVC Irradiation on FB Cells

Fibroblast cells were plated at different densities, and exposed to UVC for various time (0 s, 10 s, 60 s and 360 s), then incubated for another 24 h, 48 h or 72 h (PIT). MTT assay was used to evaluate cell viability. The assay revealed that increasing exposure times lead to decreased fibroblast viability.

### Cytotoxic Effect of Quetiapine

Quetiapine was found to have a cytotoxic effect on fibroblasts at high concentrations. Cultures were examined after 24, 48, and 72 hours after treatment with 0, 0.01, 0.1, 10, or 100 μM of Quetiapine. Survival started becoming affective at 0.1 μM of Quetiapine and decreased further at 1, 10, and 100 μM.

### UVC Exposure and Quetiapine Treatment

Cells were treated with UVC and Quetipine showed that the drug did not prevent UVC induced cytotoxicity regardless of the dose given (0, 0.01, 0.1, and 1 μM).

UVB Irradiation on Fibroblast Cells, Viability and proCOL1A1 Expression

Fibroblasts were exposed to varying intensities of UVB, 0, 20, 40 and 60 mJ/cm² and were examined 24, 48 or 72 hours after. It was found that cell survival decreases as exposure intensity increases. Examination at 48 hours post irradiation showed an increased intensity of UVB lead to an increase in MMP-1 expression and a decrease in proCOL1A1 expression. MMP-1 or matrix metalloproteinase-1 is an enzyme that is involved in the breakdown of the extracellular matrix in normal physiological processes such as embryonic development, reproduction and tissue modeling. It is also responsible for breaking down collagen. proCOL1A1 is one component that makes up a molecule of type 1 collagen, which is found in most connective tissue, including the skin.

### UVB and Quetiapine Treatment

Quetiapine treatment of fibroblasts was found not to alleviate UVB induced cell viability as evaluated by MTT assay.

Cells stained with PI-Hoechst 33342 after treatment with UVB and Quetiapine for 48 hours showed that the drug had no effect on cell death.

Quetiapine (0.1 μM) was found to significantly increase proCOL1A 1 content in cell lysates after irradiated by UVB (20 mJ/cm²) compared to UVB control cells.

### Example 2

Effects of Quetiapine Treatment on UV Exposed Skin

### Purpose

To conduct a small pilot study to examine the effect of quetiapine treatment on the skin of hairless mice to determine if it can prevent the damage associated with chronic UV exposure.

### Subjects

Ten female SKH-1 mice were obtained from Jackson Laboratory. They were 9 weeks old at the start of the experiment.

### Method

Animals were ordered at 7 weeks of age and half were pretreated with quetiapine (10 mg/kg dissolved in drinking water) for two weeks prior to commencement of UV exposure. The other half were left untreated. Quetiapine was obtained in powder form (batch 1677) from Avlon Works, in Bristol, England, a production site for the pharmaceutical company AstraZeneca. All mice were exposed three times a week for a total of 10 weeks. Exposure intensity was 90 mJ/cm and exposure duration was 7.5 minutes. In the literature, this exposure intensity and duration has been shown to produce results similar to mild sunburn. Excessive damage to the skin was not the goal of this study. The lamp used was manufactured by UVP (Upland, Calif., model UVLM-28).

Animals were free moving in a standard cage, with the light positioned about 30 cm above them. The intensity was measured and animals were not placed under the light until it had reached a stable value.

### Results

Skin after Exposure

Visual examination of the skin after 2 weeks of UV treatment showed that the skin of quetiapine treated mice had less redness, and less irritation than the skin of untreated mice.

### Histology

Skin samples were taken from the dorsal region (along the midline of the mouse close to the hindlimbs) of each mouse using a 6 mm dermal biopsy punch. For comparison purposes an additional sample was taken from skin just above the hindlimb, close to the site of damage that did not show obvious external signs of damage.

Three histological stains were used. Each stain was prepared by the histology technician in the Department of Human Anatomy and Cell Science at the University of Manitoba. Hematoxylin and Eosin (H&E) was used to examine skin structure. Van Gieson stain was used to examine collagen structure, and Verhoeff-Van Gieson stain was used to examine both collagen and elastin fibres.

Skin samples from all animals were embedded in paraffin wax blocks using standard techniques and sectioned at a thickness of 5 microns.

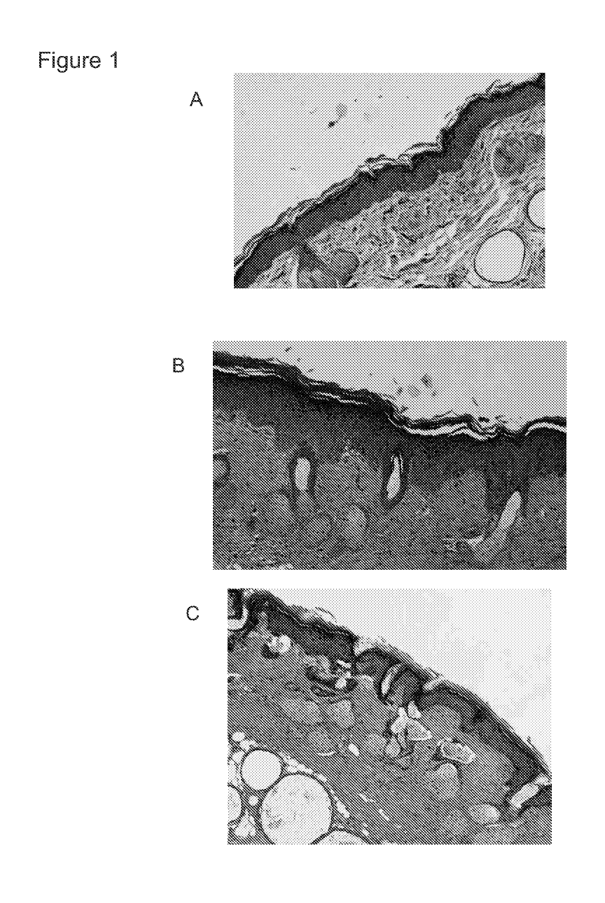

Staining with Hematoxylin and Eosin. Examination of the epidermis between the three groups showed increased thickness in the untreated group compared to control and quetiapine treated animals (FIG. 1).

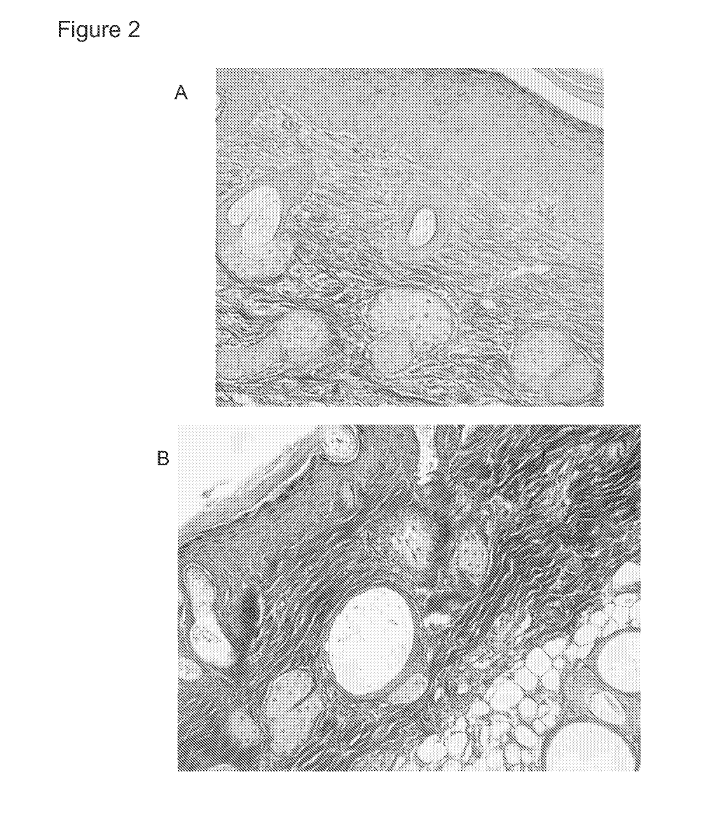

Staining with Van Gieson. The untreated group shows lighter staining and a disorganized, fragmented appearance of collagen. Treated group shows darker staining and uniform, intact appearance of collagen bands (FIG. 2).

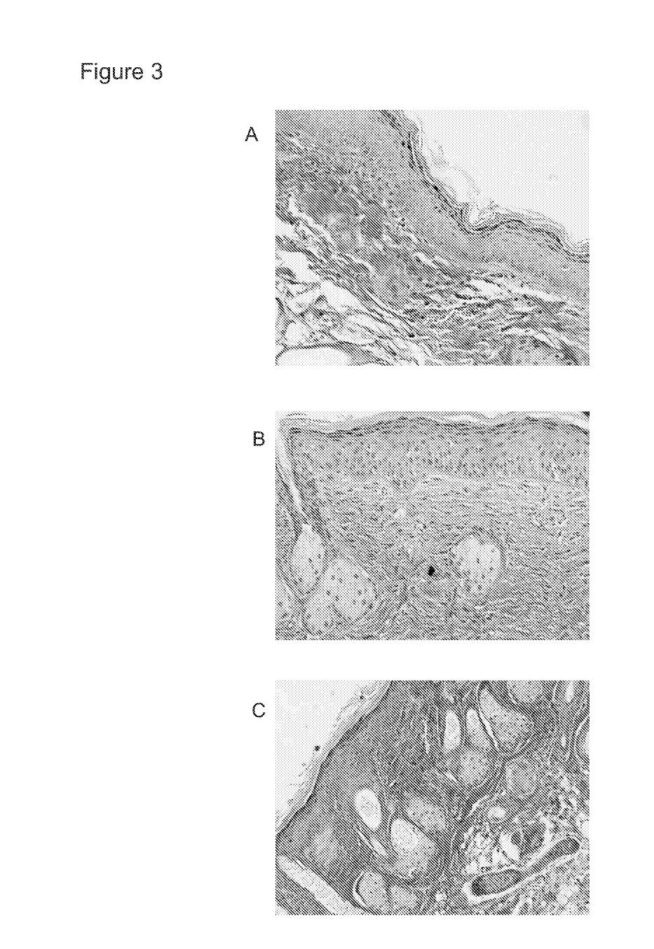

Staining with Verhoeff-Van Gieson. Fewer elastic fibres can be observed in untreated skin and those that are present are abnormally long and thin. The skin from treated animals shows better preservation of elastic fibre number and structure (FIG. 3).

### SUMMARY

Collagen and elastic fibres are both very important in maintaining the youthful appearance of skin. Collagen controls the strength, structure, firmness and overall appearance of the skin. Breakdown or loss of collagen results in a reduction in firmness, leading to the formation of wrinkles. Elastic fibres enable skin to return to its natural position after being contorted. Loss of elastic fibres causes skin to be loose and lax. Ultraviolet radiation causes collagen to break down at a higher rate than with just chronological aging. Sunlight damages collagen fibres and causes the accumulation of abnormal elastic fibres, both of which lead to pronounced changes in the outward appearance of the skin. Treatment with quetiapine appears to prevent UV induced collagen breakdown and the formation of abnormal elastic fibres in chronically exposed mice. Protection of collagen and elastic fibres helps to maintain the outward appearance of the skin, preventing the premature aging associated with UV radiation.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A method for treating photoaging comprising:
administering to a subject in need thereof an effective amount of a composition comprising an active quetiapine compound selected from quetiapine, an analog of quetiapine, and a pharmaceutically acceptable salt, solvate, and prodrug thereof, wherein the active quetiapine compound is effective in treating photoaging.

2. The method of claim 1 wherein the subject is a human.

3. The method of claim 1 wherein the administering occurs before extended exposure of the subject to a source of ultraviolet radiation.

4. The method of claim 1 wherein the administering occurs during extended exposure of the subject to a source of ultraviolet radiation.

5. The method of claim 1 wherein the treating comprises reduced redness of the subject's skin after exposure of the subject to a source of UV radiation compared to skin of the subject not administered the composition and exposed to the source of ultraviolet radiation.

6. The method of claim 1 wherein the treating comprises minimizing an increase in epidermis of the subject's skin, maintaining uniform and intact collagen bands in the subject's skin, preserving elastic fiber number and structure in the subject's skin, or a combination thereof, after exposure of the subject to a source of UV radiation compared to skin of the subject not administered the composition and exposed to the source of ultraviolet radiation.

7.-8. (canceled)

9. The method of claim 1 wherein the administering comprises use of a topical preparation.

10. (canceled)

11. The method of claim 1 wherein the administering is oral.

12.-14. (canceled)

15. The method of claim 1 wherein the active quetiapine compound is selected from a compound of Formula I, and a pharmaceutically acceptable salt and solvate thereof: 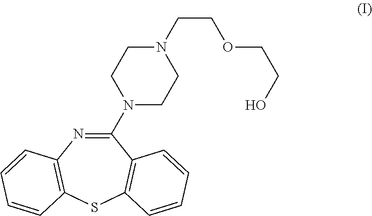 [SMILES: OCCOCCN1CCN(C2=Nc3ccccc3Sc3ccccc32)CC1]
wherein one or more hydrogen-bearing carbon atoms in the quetiapine is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

16. The method of claim 1 wherein the analog of quetiapine is selected from a compound of Formula II, and a pharmaceutically acceptable salt, solvate, and prodrug thereof: 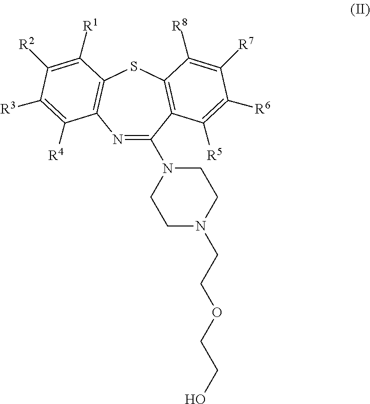
wherein any one or more of R₁, R₂, R₃, R₄, R₅, R₆, R₇, R₈ (e.g., one, two, three, four, five, six, seven, or eight) are independently selected from halogen (e.g., F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), CF₃, OCF₃CH₃, or a C1-C10 organic group (e.g., in some embodiments a C1-C4 organic group or moiety), with each R independently being hydrogen or an organic group.

17. A method for treating skin cancer comprising:
administering to a subject in need thereof an effective amount of a composition comprising an active quetiapine compound selected from quetiapine, an analog of quetiapine, and a pharmaceutically acceptable salt, solvate, and prodrug thereof, wherein the active quetiapine compound is effective in treating skin cancer.

18. The method of claim 17 wherein the subject is a human.

19. The method of claim 17 wherein the administering occurs before extended exposure of the subject to a source of ultraviolet radiation.

20.-22. (canceled)

23. The method of claim 17 wherein the administering comprises use of a topical preparation.

24. (canceled)

25. The method of claim 17 wherein the administering is oral.

26.-28. (canceled)

29. The method of claim 17 wherein skin cells of the subject have greater levels of proCOL1A1 after exposure to a source of ultraviolet radiation than skin cells of the subject not administered the composition and exposed to the source of ultraviolet radiation.

30. The method of claim 17 wherein skin cells of the subject have reduced levels of MMP1 after exposure to a source of ultraviolet radiation than skin cells of the subject not administered the composition and exposed to the source of ultraviolet radiation.

31. The method of claim 17 wherein the active quetiapine compound is selected from a compound of Formula I, and a pharmaceutically acceptable salt and solvate thereof: 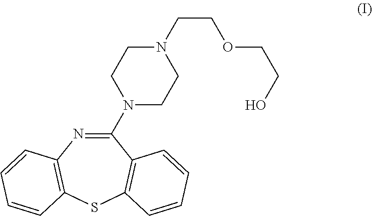 [SMILES: OCCOCCN1CCN(C2=Nc3ccccc3Sc3ccccc32)CC1]
wherein one or more hydrogen-bearing carbon atoms in the quetiapine is substituted, wherein each substituent is selected from a halogen, a nitrile, a hydroxy, an alkoxy (OR), a nitrate, a nitrite, a sulfate (O—SO₃R), an amino (NR₂), a nitro, a sulfonate (SO₂OR), or a C1-C10 organic group, wherein each R is independently a hydrogen or an organic group.

32. The method of claim 17 wherein the analog of quetiapine is selected from a compound of Formula II, and a pharmaceutically acceptable salt, solvate, and prodrug thereof: 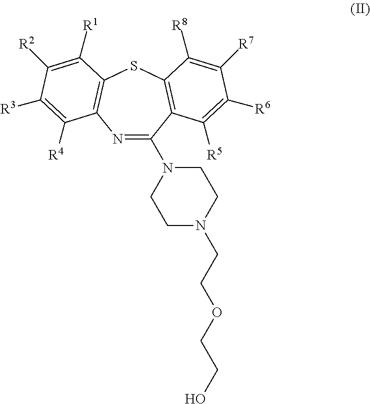
wherein any one or more of R₁, R₂, R₃, R₄, R₅, R₆, R₇, R₈ (e.g., one, two, three, four, five, six, seven, or eight) are independently selected from halogen (e.g., F, Cl, Br, I), nitrile (CN), hydroxy (OH), alkoxy (OR), nitrate (O—NO₂), nitrite (O—N═O), sulfate (O—SO₃R), amino (NR₂), nitro (NO₂), sulfonate (SO₂OR), CF₃, OCF₃CH₃, or a C1-C10 organic group (e.g., in some embodiments a C1-C4 organic group or moiety), with each R independently being hydrogen or an organic group.

33.-41. (canceled)

42. A composition comprising an active quetiapine compound selected from quetiapine, an analog of quetiapine, and a pharmaceutically acceptable salt, solvate, and prodrug thereof, and a vehicle suitable for use by epicutaneous administration.

43.-46. (canceled)
